(19) United States
(12) Patent Application Publication
(10) Pub. No.: US 2022/0328173 A1
(43) Pub. Date: Oct. 13, 2022

(54) ARTIFICIAL INTELLIGENCE BASED SYSTEM AND METHOD FOR DOCUMENTING MEDICAL PROCEDURES

(71) Applicants: Sadreddin Ramin Mahjouri, San Diego, CA (US); Vijaykumar Nayak, San Diego, CA (US)

(72) Inventors: Sadreddin Ramin Mahjouri, San Diego, CA (US); Vijaykumar Nayak, San Diego, CA (US)

(21) Appl. No.: 17/715,095

(22) Filed: Apr. 7, 2022

Related U.S. Application Data
(60) Provisional application No. 63/172,120, filed on Apr. 8, 2021.

(51) Int. Cl. G16H 40/20; G16H 30/40; G16H 50/30; G16H 50/20

(52) U.S. Cl. CPC G16H 40/20; G16H 30/40; G16H 50/30; G16H 50/20; G06V 20/41

(57) ABSTRACT

An AI-based system and method for documenting medical procedures is disclosed. The method includes receiving medical data and extracting one or more medical parameters associated with one or more medical procedures. The method includes detecting one or more vital readings of a patient and generating one or more labels for plurality of video frames, one or more images, one or more voice inputs, the one or more medical parameters, geolocation of one or more users and the detected one or more vital readings. Furthermore, the method includes annotating the one or more labels in the one or more videos and the one or more images, receiving a request to retrieve one or more specific frames and a set of specific images. The method includes retrieving and outputting the one or more specific frames and the set of specific images on one or more electronic devices.

### EARLIEST PRIORITY DATE

This application claims priority from a Provisional patent application filed in the United States of America having Patent Application No. 63/172,120, filed on Apr. 8, 2021, and titled “SYSTEM AND METHOD FOR DOCUMENTING MEDICAL PROCEDURES”.

### FIELD OF INVENTION

Embodiments of the present disclosure relate to Artificial Intelligence (AI) based systems and more particularly relates to an AI-based system and method for documenting medical procedures.

### BACKGROUND

Generally, a patient undergoes a medical procedure with an intention of determining, measuring, and/or diagnosing a health condition. While performing medical procedures on the patient, it is usually preferred by some clinicians or doctors to document the medical procedures in form of video recordings. Later, from the recorded video, it might be necessary to retrieve a particular section of the video for auditing, training, and evaluation. Video recording is performed to extract maximum information about the medical procedure. Usually, the clinician or the doctor uses a head mounted camera-based system to record the medical procedures. Any medical procedure with hierarchical subprocesses may be documented by such stated process. In a conventional approach, an evaluator or trainer goes through an entire video to understand a minute portion of the entire medical procedure. There is no efficient way to locate the point of interest of the entire procedure. Thus, the conventional approach of understanding the medical procedure consumes time and requires a lot of efforts.

Hence, there is a need for an improved AI-based system and method for documenting medical procedures, in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, an AI-based computing system for documenting medical procedures is disclosed. The AI-based computing system includes one or more hardware processors and a memory coupled to the one or more hardware processors. The memory includes a plurality of modules in the form of programmable instructions executable by the one or more hardware processors. The plurality of modules include a medical data receiver module configured to receive medical data associated with one or more medical procedures from one or more data capturing units. The medical data includes at least one of: one or more videos, one or more images of one or more medical procedures, one or more voice inputs and geolocation of one or more users. A plurality of video frames are extracted from each of the one or more videos by using a frame extraction technique. The plurality of modules also include a medical parameter extraction module configured to extract one or more medical parameters associated with the one or more medical procedures from at least one of: the plurality of video frames and the one or more images by using a document management-based AI model. The plurality of modules includes a vital detection module configured to detect one or more vital readings of a patient during the one or more medical procedures by using one or more sensors. The one or more vital readings include: blood pressure, pulse, temperature and respiration rate. Further, the plurality of modules includes a label generation module configured to generate one or more labels for each of: the plurality of video frames, the one or more images, the one or more voice inputs, the extracted one or more medical parameters, the geolocation of one or more users and the detected one or more vital readings based on predefined label information and predefined registration information by using the document management-based AI model. The plurality of modules also include an annotation module configured to annotate the generated one or more labels in the one or more videos and the one or more images by correlating the plurality of video frames, the one or more images, the one or more voice inputs, the extracted one or more medical parameters, the geolocation of one or more users and the detected one or more vital readings based on their corresponding one or more timestamps by using the document management-based AI model. Furthermore, the plurality of modules include a request receiver module configured to receive a request from the one or more users to retrieve at least one of: one or more specific frames from the annotated one or more videos and a set of specific images from the annotated one or more images. The received request includes: one or more keywords corresponding to at least one of: the plurality of video frames, the one or more images, the one or more voice inputs, the extracted one or more medical parameters, the predefined registration information, the geolocation of one or more users and the detected one or more vital readings. The plurality of modules include a data retriever module configured to retrieve at least one of: the one or more specific frames from the annotated one or more videos and the set of specific images from the annotated one or more images based on the generated one or more labels and the one or more keywords by using the document management-based AI model. The plurality of modules include a data output module configured to output the retrieved at least one of: the one or more specific frames and the set of specific images on user interface screen of one or more electronic devices associated with the one or more users.

In accordance with another embodiment of the present disclosure, an AI-based method for documenting medical procedures is disclosed. The AI-based method includes receiving medical data associated with one or more medical procedures from one or more data capturing units. The medical data includes at least one of: one or more videos, one or more images of one or more medical procedures, one or more voice inputs and geolocation of one or more users. A plurality of video frames are extracted from each of the one or more videos by using a frame extraction technique. The AI-based method further includes extracting one or more medical parameters associated with the one or more medical procedures from at least one of: the plurality of video frames and the one or more images by using a document management-based AI model. Further, the AI-based method includes detecting one or more vital readings of a patient during the one or more medical procedures by using one or more sensors. The one or more vital readings include: blood pressure, pulse, temperature and respiration rate. Also, the AI-based method includes generating one or more labels for each of: the plurality of video frames, the one or more images, the one or more voice inputs, the extracted one or more medical parameters, the geolocation of one or more users and the detected one or more vital readings based on predefined label information and predefined registration information by using the document management-based AI model. Furthermore, the AI-based method includes annotating the generated one or more labels in the one or more videos and the one or more images by correlating the plurality of video frames, the one or more images, the one or more voice inputs, the extracted one or more medical parameters, the geolocation of one or more users and the detected one or more vital readings based on their corresponding one or more timestamps by using the document management-based AI model. The AI-based method also includes receiving a request from the one or more users to retrieve at least one of: one or more specific frames from the annotated one or more videos and a set of specific images from the annotated one or more images. The received request includes: one or more keywords corresponding to at least one of: the plurality of video frames, the one or more images, the one or more voice inputs, the extracted one or more medical parameters, the predefined registration information, the geolocation of one or more users and the detected one or more vital readings. Further, the AI-based method includes retrieving at least one of: the one or more specific frames from the annotated one or more videos and the set of specific images from the annotated one or more images based on the generated one or more labels and the one or more keywords by using the document management-based AI model. The method includes outputting the retrieved at least one of: the one or more specific frames and the set of specific images on user interface screen of one or more electronic devices associated with the one or more users.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 is a block diagram illustrating an exemplary computing environment for documenting medical procedures, in accordance with an embodiment of the present disclosure;

FIG. 2 is a block diagram illustrating an exemplary AI-based computing system for documenting medical procedures, in accordance with an embodiment of the present disclosure;

FIG. 3A is a block diagram illustrating an exemplary data retrieval module, in accordance with an embodiment of the present disclosure;

FIG. 3B is a block diagram illustrating an exemplary medical parameter extraction module, in accordance with an embodiment of the present disclosure;

FIGS. 3C-3E are block diagrams illustrating an exemplary operation of the AI-based computing system for documenting medical procedures, in accordance with an embodiment of the present disclosure; and

FIG. 4 is a process flow diagram illustrating an exemplary AI-based method for documenting medical procedures, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

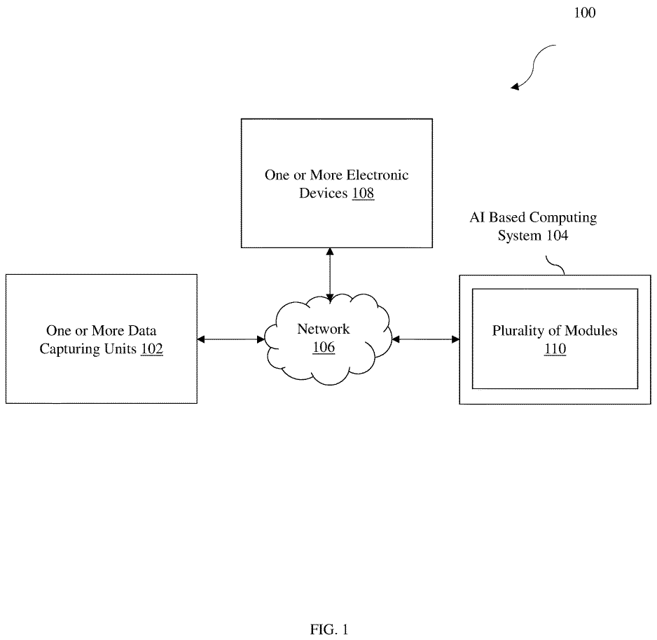

Referring now to the drawings, and more particularly to FIG. 1 through FIG. 4, where similar reference characters denote corresponding features consistently throughout the figures, there are shown preferred embodiments and these embodiments are described in the context of the following exemplary system and/or method.

FIG. 1 is a block diagram illustrating an exemplary computing environment 100 for documenting medical procedures, in accordance with an embodiment of the present disclosure. According to FIG. 1, the computing environment 100 includes one or more data capturing units 102 communicatively coupled to an AI-based computing system 104 via a network 106. The one or more data capturing units 102 are configured to capture medical data associated with one or more medical procedures. In an embodiment of the present disclosure, the one or more medical procedures are performed with the intention of determining, measuring, or diagnosing a patient's medical condition. In an exemplary embodiment of the present disclosure, the medical data includes one or more videos, one or more images of one or more medical procedures, one or more voice inputs, geolocation of one or more users or any combination thereof. In an exemplary embodiment of the present disclosure, the one or more users are one or more health professionals performing the one or more medical procedures, such as operating doctors, nursing staff associated with the one or more medical procedures, and the like. In an embodiment of the present disclosure, the one or more data capturing units 102 include one or more image capturing units, one or more audio capturing units, one or more Global Positioning System (GPS) units and the like. For example, the one or more audio capturing units include a single or multi-channel distributed microphone on a headset. In an exemplary embodiment of the present disclosure, the one or more image capturing units include one or more infrared video cameras, Red Green Blue (RGB) video camera, time-of-flight camera, one or more Three-Dimensional (3D) sensor for 3D data acquisition or any combination thereof. The network 106 may be internet or any other wireless network. The computing system may be hosted on a central server, such as cloud server or a remote server.

Further, the computing environment 100 includes one or more electronic devices 108 associated with the one or more users communicatively coupled to the AI-based computing system 104 via the network 106. The one or more electronic devices 108 are used by the one or more users to request the AI-based computing system 104 to retrieve one or more specific frames from annotated one or more videos and a set of specific images from annotated one or more images. Furthermore, the one or more electronic devices 108 are also used by the one or more users to receive the retrieved one or more specific frames and the retrieved set of specific images. In an exemplary embodiment of the present disclosure, the one or more electronic devices 108 may include a laptop computer, desktop computer, tablet computer, smartphone, wearable device, smart watch, a digital camera and the like.

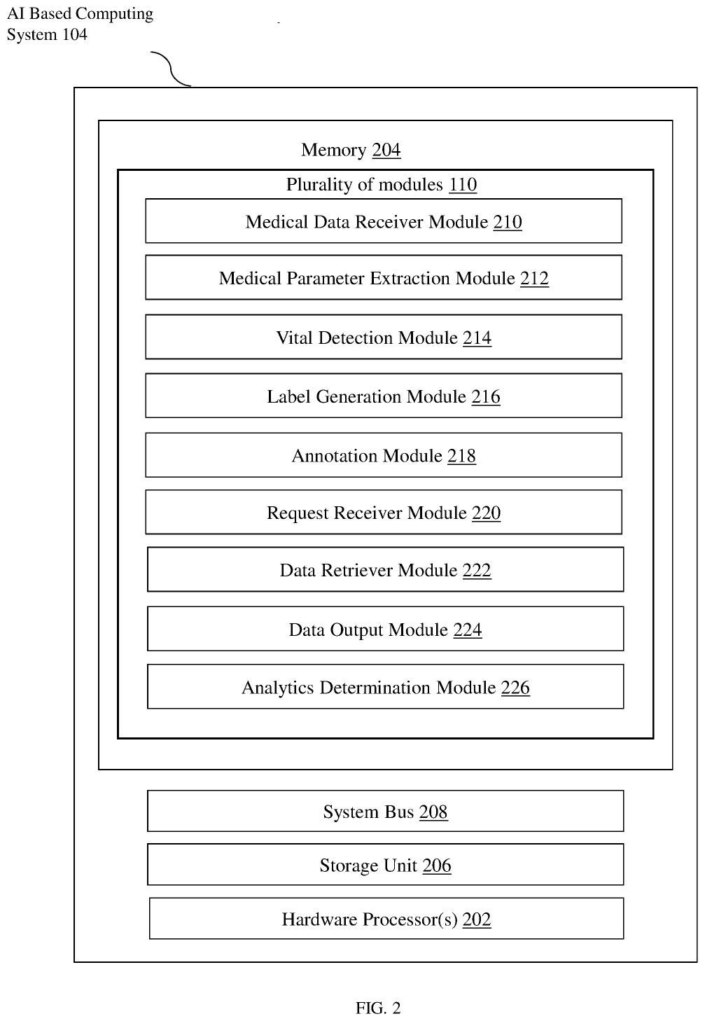

Further, the one or more electronic devices 108 include a local browser, a mobile application or a combination thereof. Furthermore, the one or more users may use a web application via the local browser, the mobile application or a combination thereof to communicate with the AI-based computing system 104. In an exemplary embodiment of the present disclosure, the mobile application may be compatible with any mobile operating system, such as android, iOS, and the like. In an embodiment of the present disclosure, the AI-based computing system 104 includes a plurality of modules 110. Details on the plurality of modules have been elaborated in subsequent paragraphs of the present description with reference to FIG. 2.

In an embodiment of the present disclosure, the AI-based computing system 104 is configured to receive the medical data associated with the one or more medical procedures from the one or more data capturing units 102. Further, plurality of video frames are extracted from each of the one or more videos by using a frame extraction technique. The AI-based computing system 104 extracts one or more medical parameters associated with the one or more medical procedures from the plurality of video frames, the one or more images or a combination thereof by using a document management-based AI model. The AI-based computing system 104 detects one or more vital readings of the patient during the one or more medical procedures by using one or more sensors. Furthermore, the AI-based computing system 104 generates one or more labels for each of the plurality of video frames, the one or more images, the one or more voice inputs, the extracted one or more medical parameters, the geolocation of one or more users and the detected one or more vital readings based on predefined label information and predefined registration information by using the document management-based AI model. The AI-based computing system 104 annotates the generated one or more labels in the one or more videos and the one or more images by correlating the plurality of video frames, the one or more images, the one or more voice inputs, the extracted one or more medical parameters, the geolocation of one or more users and the detected one or more vital readings based on their corresponding one or more timestamps by using the document management-based AI model. The AI-based computing system 104 receives a request from the one or more users to retrieve the one or more specific frames from the annotated one or more videos, the set of specific images from the annotated one or more images or a combination thereof. Further, the AI-based computing system 104 retrieves the one or more specific frames from the annotated one or more videos, the set of specific images from the annotated one or more images or a combination thereof based on the generated one or more labels and one or more keywords by using the document management-based AI model. The AI-based computing system 104 outputs the retrieved one or more specific frames, the retrieved set of specific images or a combination thereof on user interface screen of the one or more electronic devices 108 associated with the one or more users.

FIG. 2 is a block diagram illustrating an exemplary AI-based computing system for documenting medical procedures, in accordance with an embodiment of the present disclosure. Further, the AI-based computing system 104 includes one or more hardware processors 202, a memory 204 and a storage unit 206. The one or more hardware processors 202, the memory 204 and the storage unit 206 are communicatively coupled through a system bus 208 or any similar mechanism. The memory 204 comprises the plurality of modules 110 in the form of programmable instructions executable by the one or more hardware processors 202. Further, the plurality of modules 110 includes a medical data receiver module 210, a medical parameter extraction module 212, a vital detection module 214, a label generation module 216, an annotation module 218, a request receiver module 220, a data retriever module 222, a data output module 224 and an analytics determination module 226. According to some embodiments, the label generation module is controlled by an external device such as a foot paddle where the doctor or clinician can press to identify the point of interest and label it later at their desk using the computer for future references.

The one or more hardware processors 202, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 202 may also include embedded controllers, such as generic or programmable logic devices or arrays, application specific integrated circuits, single-chip computers, and the like.

The memory 204 may be non-transitory volatile memory and non-volatile memory. The memory 204 may be coupled for communication with the one or more hardware processors 202, such as being a computer-readable storage medium. The one or more hardware processors 202 may execute machine-readable instructions and/or source code stored in the memory 204. A variety of machine-readable instructions may be stored in and accessed from the memory 204. The memory 204 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 204 includes the plurality of modules 110 stored in the form of machine-readable instructions on any of the above-mentioned storage media and may be in communication with and executed by the one or more hardware processors 202.

The storage unit 206 may be a cloud storage or local storage. The storage unit 206 may store the received medical data, the one or more medical parameters, the plurality of video frames, the one or more vital readings and the generated one or more labels. The storage unit 206 may also store the predefined label information, the predefined registration information, the one or more timestamps, predefined voice information and the one or more keywords. In an embodiment of the present disclosure, the storage unit 206 is necessary for further evaluation of the one or more medical procedures or training of new medical practitioners.

The medical data receiver module 210 is configured to receive the medical data associated with the one or more medical procedures from the one or more data capturing units 102. In an embodiment of the present disclosure, the one or more medical procedures are performed with the intention of determining, measuring, or diagnosing a patient's medical condition. In an exemplary embodiment of the present disclosure, the medical data includes one or more videos, one or more images of one or more medical procedures, one or more voice inputs, geolocation of one or more users or any combination thereof. The one or more videos may be infrared videos, Red Green Blue (RGB) videos, depth videos and the like. In an embodiment of the present disclosure, the one or more images may be one or more image frames. In an exemplary embodiment of the present disclosure, the one or more users are one or more health professionals performing the one or more medical procedures, such as operating doctors, nursing staff associated with the one or more medical procedures, and the like. In an embodiment of the present disclosure, the one or more data capturing units 102 include one or more image capturing units, one or more audio capturing units, one or more Global Positioning System (GPS) units and the like. The one or more GPS units facilitates determination of exact location of medical hospital where the one or more medical procedures are taking place. The one or more image capturing units and the one or more audio capturing units are affixed at multiple places to capture the one or more medical procedures efficiently from multiple angles. In an embodiment of the present disclosure, the one or more image capturing units may be one or more video capturing units. For example, the one or more audio capturing units include single or multi-channel distributed microphone on a headset. In an exemplary embodiment of the present disclosure, the one or more image capturing units include one or more infrared video cameras, RGB video camera, time-of-flight camera, one or more Three-Dimensional (3D) sensor for 3D data acquisition or any combination thereof. In an embodiment of the present disclosure, high-resolution stream is stored in a local storage and transmitted to a server in background for storage in a remote database. Also, video stream associated with the one or more videos, or the one or more images are visualized live (in real-time) wirelessly on a monitor or mobile platform by down sampling appropriately via a video BW limiter. In an embodiment of the present disclosure, the medical data receiver module 210 processes the one or more videos for low latency, such that important information may be extracted from the processed one or more videos. Further, a plurality of video frames are extracted from each of the one or more videos.

In an embodiment of the present disclosure, the one or more voice inputs may be pretrained on one or more health professionals' voice, even through one or more masks, to reject spurious commands from other doctors, clinicians or nurses. The presence of multiple microphones on the headset may be used for beamforming, thereby rejecting spurious noise from the surroundings.

The medical parameter extraction module 212 configured to extract the one or more medical parameters associated with the one or more medical procedures from the plurality of video frames, the one or more images or a combination thereof by using a document management-based AI model. In an embodiment of the present disclosure, the document management-based AI model may be a machine learning model. In an exemplary embodiment of the present disclosure, the one or more medical parameters include a set of hand gesture movements of the one or more users, one or more operating tools used by the one or more users and number of the one or more users. In an embodiment of the present disclosure, the medical parameter extraction module 212 extracts and stores hand gesture movement details with corresponding associated voice inputs.

The vital detection module 214 is configured to detect the one or more vital readings of the patient during the one or more medical procedures by using the one or more sensors. In an exemplary embodiment of the present disclosure, the one or more vital readings include blood pressure, pulse, temperature, respiration rate and the like. For example, the one or more sensors include pressure sensor, force sensor, airflow sensor, pulse oximetry, temperature sensor and the like.

The label generation module 216 is configured to generate the one or more labels for each of the plurality of video frames, the one or more images, the one or more voice inputs, the extracted one or more medical parameters, the geolocation of one or more users and the detected one or more vital readings based on predefined label information and predefined registration information by using the document management-based AI model. In an embodiment of the present disclosure, the predefined registration information is entered by the one or more users at time of registering the patient and the one or more health professionals with the AI-based computing system 104 before the one or more medical procedures. In an exemplary embodiment of the present disclosure, the predefined registration information include patient name, patient address, patient medical history data, medical procedure details, medical professional details and the like. For example, the medical procedure details include name of the medical procedure, number of medical procedures required to diagnose the patient and the like. For example, the medical professional details include name, designation, type of the one or more health professionals and the like. In an exemplary embodiment of the present disclosure, the type of the one or more health professionals include doctor, nursing staff and the like. In generating the one or more labels for each of the plurality of video frames, the one or more images, the one or more voice inputs, the extracted one or more medical parameters, the geolocation of one or more users and the detected one or more vital readings based on predefined label information and predefined registration information by using the document management-based AI model, the label generation module 216 converts the one or more voice inputs into one or more text outputs by using the document management-based AI model. Further, the label generation module 216 identifies a set of relevant keywords from the one or more text outputs based on the predefined registration information and the predefined label information by using the document management-based AI model. The label generation module 216 identify speaker of each of the one or more voice inputs based on predefined voice information by using the document management-based AI model. In an embodiment of the present disclosure, the predefined voice information include waveform, behavior characteristics associated with the one or more users and the like. Furthermore, the label generation module 216 generates the one or more labels corresponding to the one or more voice inputs based on the identified set of relevant keywords, the identified speaker, the predefined registration information and the predefined label information by using the document management-based AI model. In an embodiment of the present disclosure, the plurality of video frames, the one or more images, the one or more voice inputs and the one or more medical parameters are labelled as points of interest by a strategic two dimensional (2D) labelled marker. In an embodiment of the present disclosure, the label generation module 216 is the strategic 2D labelled marker. This labelling is required to extract the interested point of view at a future date. Further, such data augmentation enables a user to minimize a large video recording to a relatively smaller video file.

The annotation module 218 is configured to annotate the generated one or more labels in the one or more videos and the one or more images by correlating the plurality of video frames, the one or more images, the one or more voice inputs, the extracted one or more medical parameters, the geolocation of one or more users and the detected one or more vital readings based on their corresponding one or more timestamps by using the document management-based AI model. In annotating the generated one or more labels in the one or more videos and the one or more images by correlating the plurality of video frames, the one or more images, the one or more voice inputs, the extracted one or more medical parameters, the geolocation of one or more users and the detected one or more vital readings based on their corresponding one or more timestamps by using the document management-based AI model, the annotation module 218 correlates the one or more voice inputs, the extracted one or more medical parameters, the geolocation of one or more users and the detected one or more vital readings with the plurality of video frames and the one or more images based on the one or more timestamps by using the document management-based AI model. Further, the annotation module 218 annotates the generated one or more labels in the one or more videos and the one or more images upon performing correlation.

The request receiver module 220 is configured to receive the request from the one or more users to retrieve the one or more specific frames from the annotated one or more videos, the set of specific images from the annotated one or more images or a combination thereof. In an embodiment of the present disclosure, the received request includes one or more keywords corresponding to the plurality of video frames, the one or more images, the one or more voice inputs, the extracted one or more medical parameters, the predefined registration information, the geolocation of one or more users, the detected one or more vital readings or any combination thereof. In an embodiment of the present disclosure, the one or more keywords refer to words describing specific context. For example, when the one or more users require to retrieve a portion of a video in which heart rate of the patient is 110, the one or more users may use “heart rate above 110” as the one or more keywords.

In an embodiment of the present disclosure, the one or more keywords are associated with the one or more labels generated for the one or more voice inputs. For example, when the one or more users require to retrieve a portion of a video in which a doctor said perform the incision, the one or more users may use “perform the incision” as the one or more keywords.

The data retriever module 222 is configured to retrieve the one or more specific frames from the annotated one or more videos, the set of specific images from the annotated one or more images or a combination thereof based on the generated one or more labels and the one or more keywords by using the document management-based AI model. In retrieving the one or more specific frames from the annotated one or more videos, the set of specific images from the annotated one or more images or a combination thereof based on the generated one or more labels and the one or more keywords by using the document management-based AI model, the data retriever module 222 compares the one or more keywords with the generated one or more labels in the annotated one or more videos, the annotated one or more images or a combination thereof by using the document management-based AI model. Further, the data retriever module 222 retrieves the one or more specific frames from the annotated one or more videos, the set of specific images from the annotated one or more images or a combination thereof based on result of comparison. The data retriever module 222 retrieves a set of voice inputs, a set of medical parameters, the geolocation of one or more users and a set of one or more vital readings corresponding to the retrieved one or more specific frames, the retrieved set of specific images or a combination thereof. In an embodiment of the present disclosure, the retrieved one or more specific frames, the retrieved set of specific images or a combination thereof, the retrieved set of voice inputs, the retrieved set of medical parameters, the retrieved geolocation of one or more users and the retrieved set of one or more vital readings are outputted on user interface screen of the one or more electronic devices 108 associated with the one or more users via one or more output formats. For example, the one or more output formats include audio, image, text, video and the like.

In an embodiment of the present disclosure, the data retriever module 222 extracts and stores the one or more voice inputs and associated recorded video reference in the storage unit 206. The one or more voice inputs may be associated with one or more operating doctors. In an embodiment of the present disclosure, the data retriever module 222 searches for a video frame or image in order to provide an image or a video query. The AI-based computing system 104 also down-samples data, performs segmentation on the one or more images or the plurality of video frames to detect point of interest in the one or more images or the plurality of video frames.

For example, a specialist doctor may be performing laparoscopy for pancreatic cancer. The process generally requires small incisions. A user may define the point of interest on the keyword “incision” by using the AI-based computing system 104. The AI-based computing system 104 may extract a video frame with the incision word. Augment stored voice inputs as captured by microphone with the extracted video frame. The hand gesture details of the doctor are also noted from the video frame. All such details are synced and stored for viewing in the storage unit 206.

The data output module 224 is configured to output the retrieved the one or more specific frames, the set of specific images or a combination thereof on user interface screen of the one or more electronic devices 108 associated with the one or more users. In an exemplary embodiment of the present disclosure, the one or more electronic devices 108 may include a laptop computer, desktop computer, tablet computer, smartphone, wearable device, smart watch, a digital camera and the like. In an embodiment of the present disclosure, outputting the one or more specific frames, the set of specific images or a combination thereof on user interface screen of the one or more electronic devices 108 enables easy detection without need for going through whole captured video.

In an embodiment of the present disclosure, before using the document management-based AI model for performing multiple operations, such as label generation, annotation and the like, the document management-based AI model is required to be trained. Further, the document management-based AI model is pretrained on a dataset of such operating procedure videos with commentary metadata and mixture of supervised and unsupervised video data training. In an embodiment of the present disclosure, tools are annotated in training video and hand pose detections are refined using existing models. Thus, this step drastically improves search speeds through huge database of videos. Furthermore, the AI-based computing system 104 creates a searchable representation of the videos, thereby stores the data as a metadata for procedure in a hierarchical representation.

The analytics determination module 226 determines one or more analytics parameters associated with the one or more medical procedures by analyzing the medical data using the document management-based AI model. In an embodiment of the present disclosure, the one or more analytics parameters include time consumed in each stage of the one or more medical procedures, response of the patient to certain operating events and the like. In an embodiment of the present disclosure, time consumed in each stage of the one or more medical procedures is associated with the one or more vital readings of the patient to ascertain patient response to certain events. For example, response of the patient to certain operation event may be fluctuation in heart rate, sudden decrease in SpO2 level and the like upon performing incision. Further, the analytics determination module 226 outputs the determined one or more analytics parameters on user interface screen of the one or more electronic devices 108 associated with the one or more users. In an embodiment of the present disclosure, the one or more users may also retrieve one or more portions of the one or more videos, the one or more images or a combination thereof corresponding to the determined one or more analytics parameters by using specific keywords related to the determined one or more analytics parameters. In an embodiment of the present disclosure, analytics associated to doctor's commentary, recorded visuals and patient vitals are also presented in real time.

In an embodiment of the present disclosure, the AI-based computing system 104 provides a search option of any operating event based on “context aware” search that first searches through explicit tags like voice recording and implicit tags like aggregated sensor data. For example, an explicit search may be done for a “root canal” procedure, but an implicit search may be done for the video when the heart rate was fluctuating during the “root canal” procedure. In another embodiment of the present disclosure, the AI-based computing system 104 enables creation of a “virtualized” or immersive experience video, thereby improving the accuracy of learning systems due to multiple perspectives of any given procedure.

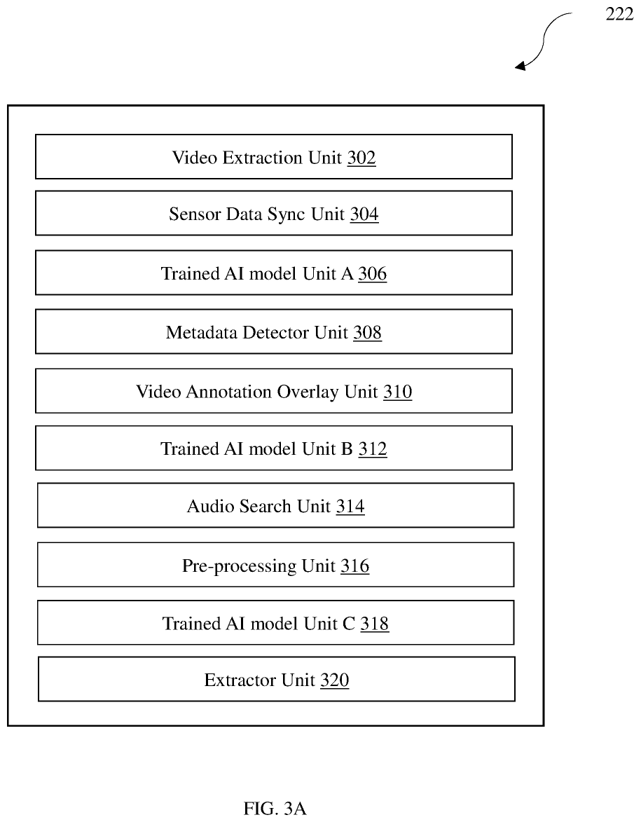

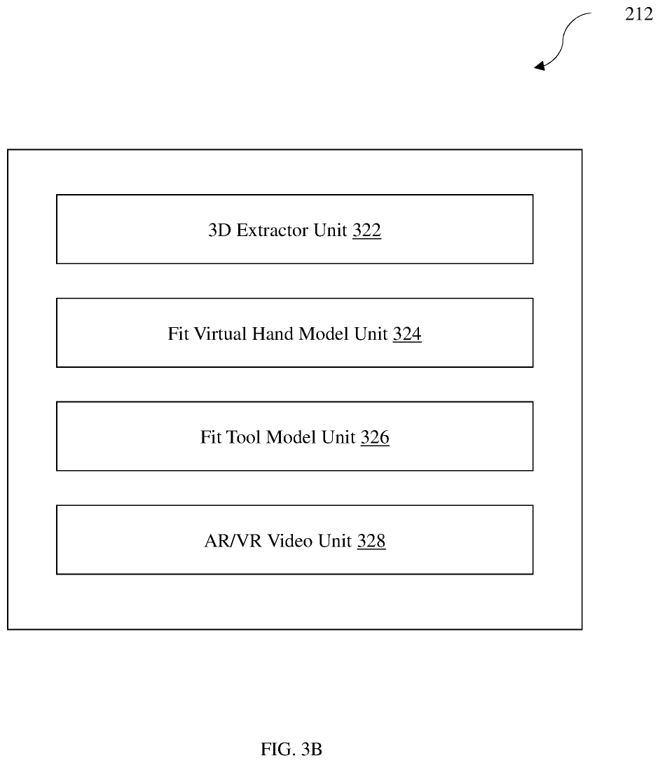

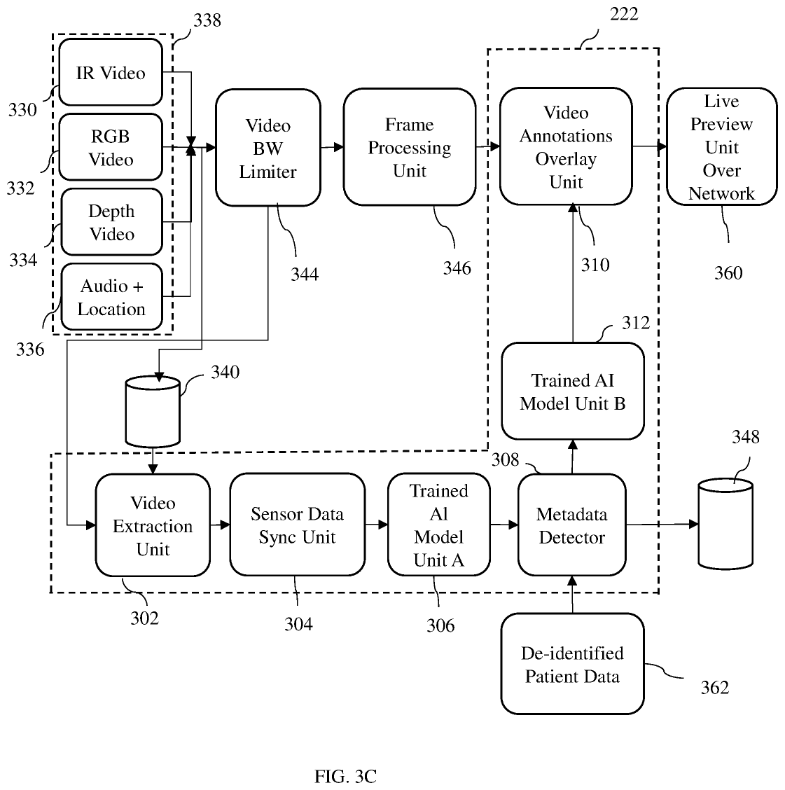

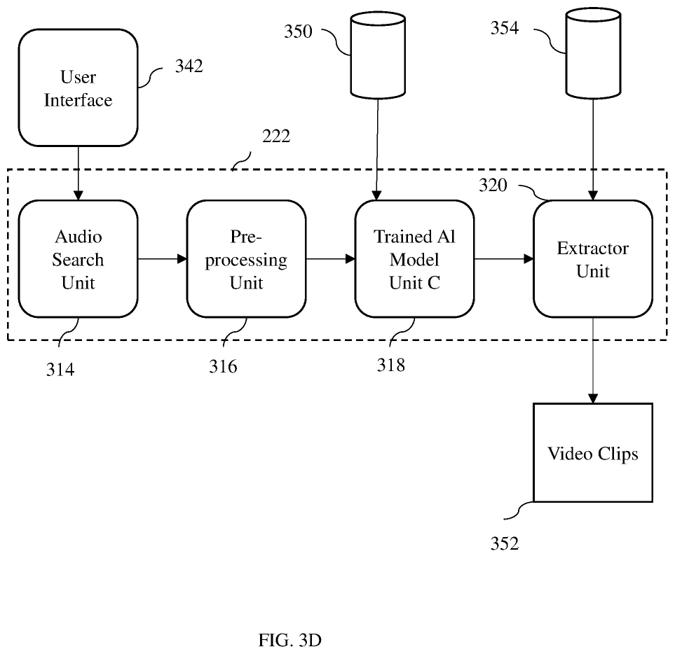

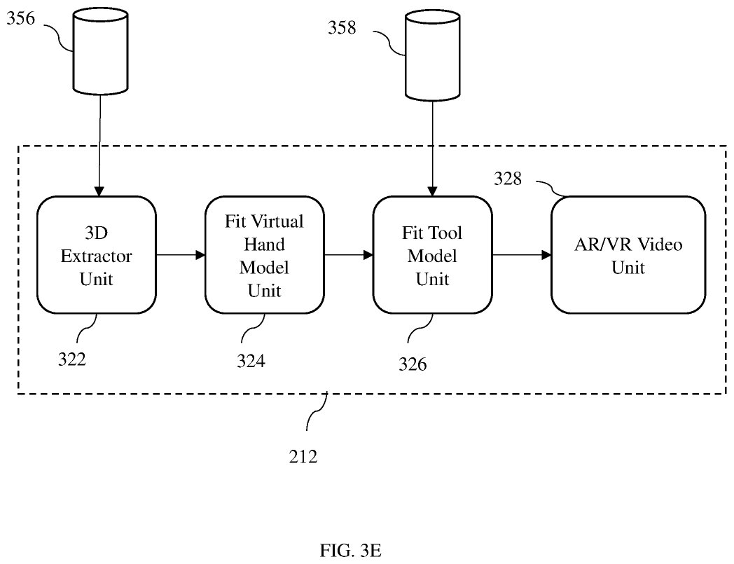

FIG. 3A is a block diagram illustrating an exemplary data retrieval module, in accordance with an embodiment of the present disclosure. Further, FIG. 3B is a block diagram illustrating an exemplary medical parameter extraction module 212, in accordance with an embodiment of the present disclosure. FIGS. 3C-3E are block diagrams illustrating an exemplary operation of the AI-based computing system 104 for documenting medical procedures, in accordance with an embodiment of the present disclosure. For the sake of brevity, FIGS. 3A-3E have been explained together.

The data retrieval module includes a video extraction unit 302, a sensor data sync unit 304, a trained AI model unit A 306, a metadata detector unit 308, a video annotation overlay unit 310, a trained AI model unit B 312, an audio search unit 314, a pre-processing unit 316, a trained AI model unit C 318 and an extractor unit 320, as shown in FIG. 3A. In an embodiment of the present disclosure, each of the trained AI model unit A 306, the trained AI model unit B 312 and the trained AI model unit C 318 may be an AI model or ML model. Further, the medical parameter extraction module 212 includes a 3D extractor unit 322, a fit virtual hand model unit 324, a fit tool model unit 326 and an AR/VR video unit 328, as shown in FIG. 3B.

In an embodiment of the present disclosure, FIG. 3C depicts workflow indicating how the one or more videos are captured and stored in the storage unit 206, such that the one or more videos may be accessed with different keywords. Further, infrared videos 330, RGB videos 332, depth videos 334 and like are captured by the one or more data capturing units 102. The one or more data capturing units 102 also captures the one or more voice inputs and geolocation of one or more users 336. In an embodiment of the present disclosure, the infrared videos 330, the RGB videos 332, the depth videos 334, the one or more voice inputs and geolocation of one or more users 336 may be called medical data 338 associated with one or more medical procedures. A file storage database unit 340 stores the medical data 338. The one or more videos or the one or more images may be viewed in real time through a user interface 342 with video BW limiter 344, as shown in FIGS. 3C and 3D. Frame processing unit 346 processes the captured one or more videos for low latency. In an embodiment of the present disclosure, the frame processing unit 346 corresponds to the media data receiver module 210. Furthermore, the video annotations overlay unit 310 extracts important information from the video.

Further, the video extraction unit 302 extracts a region of interest in accordance with the one or more keywords. In such embodiment, the sensor data sync unit 304 is configured to sync all related extracted region. The trained AI model unit A 306 help in extraction of interested region. Furthermore, the metadata detector 308 stores the extracted interested region in a storage database 348. In an embodiment of the present disclosure, the trained AI model unit B 312 helps in providing information to the video annotations overlay unit 310 based on metadata detector 308 input. Furthermore, such training systems are implemented for subsequent procedure to detect anomalies in real time. This approach also aids to show/document/present specific procedures and to identify the cause of a problem.

In an embodiment of the present disclosure, FIG. 3D depicts workflow for retrieval of a video of the one or more medical procedures. A clinician through the user interface 342 may search in a related video database 350 any region of interest. The user interface 342 may be user interface screen of the one or more electronic devices 108. In one embodiment, the clinician provides a name and the audio search unit 314 searches with the provided patient's name. The pre-processing unit 316 performs the matching of name and audio process. Based on patient metadata and query, the trained AI model unit C 318 searches the related video database 350 for related videos. The extractor unit 320 is used for extracting the detected data. Video clips 352 are extracted from stored video database 354.

Furthermore, FIG. 3E depicts workflow for extraction of augmented reality and virtual reality videos. The 3D extractor unit 322 extracts hand poses of clinicians from the videos of region of interest as stored in a first database 356. Further, the fit virtual hand model unit 324 along with the fit tool model unit 326 produces virtual hand and tools video with the help of AR/VR video unit 328. The fit tool model unit 326 uses data related to tools usage from a second storage database 358. In an embodiment of the present disclosure, the file storage database unit 340, the storage database 348, the video database 354, the related video database 350, the first database 356 and the second database 358 are stored in the storage unit 206.

In an embodiment of the present disclosure, a live preview unit over network 360 facilitates simultaneous view of the one or more images while the operation is taking place. Further, de-identified patient data 362 corresponds to ability to add more details or describe specific to the operation, such that AI model may learn for future self AI based identification.

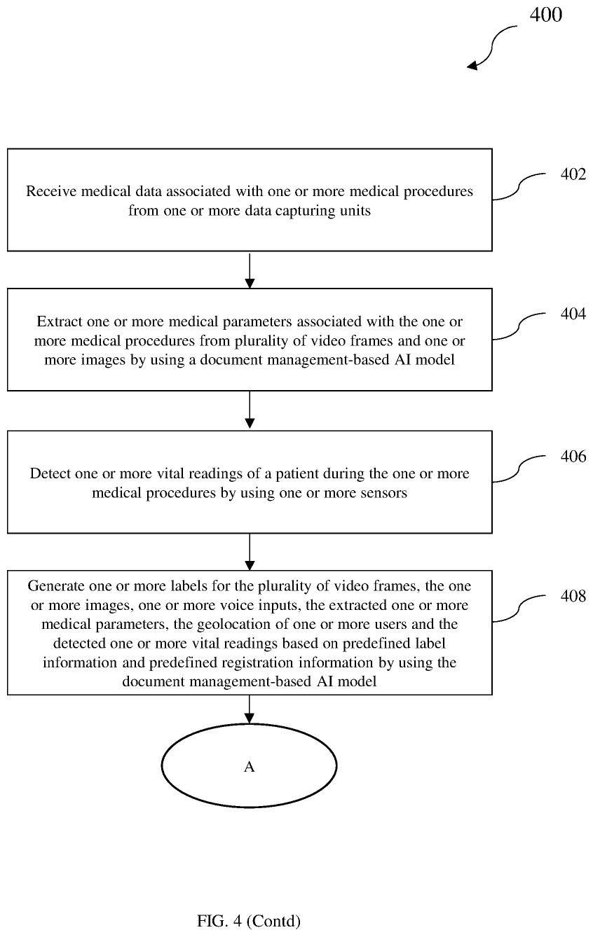

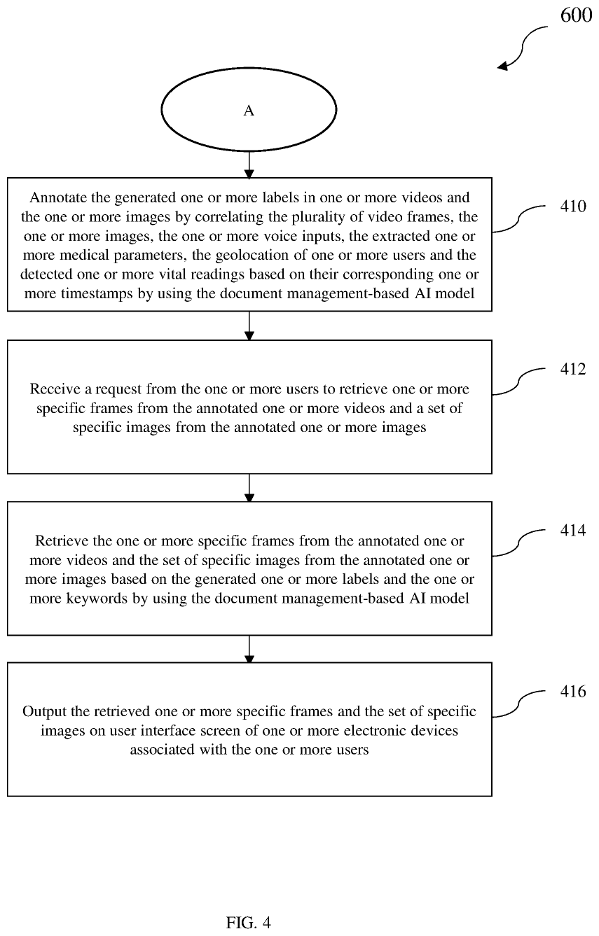

FIG. 4 is a process flow diagram illustrating an exemplary AI-based method for documenting medical procedures, in accordance with an embodiment of the present disclosure. At step 402, medical data associated with one or more medical procedures is received from the one or more data capturing units 102. In an embodiment of the present disclosure, the one or more medical procedures are performed with the intention of determining, measuring, or diagnosing a patient's medical condition. In an exemplary embodiment of the present disclosure, the medical data includes one or more videos, one or more images of one or more medical procedures, one or more voice inputs, geolocation of one or more users or any combination thereof. The one or more videos may be infrared videos, Red Green Blue (RGB) videos, depth videos and the like. In an embodiment of the present disclosure, the one or more images may be one or more image frames. In an exemplary embodiment of the present disclosure, the one or more users are one or more health professionals performing the one or more medical procedures, such as operating doctors, nursing staff associated with the one or more medical procedures, and the like. In an embodiment of the present disclosure, the one or more data capturing units 102 include one or more image capturing units, one or more audio capturing units, one or more Global Positioning System (GPS) units and the like. The one or more GPS units facilitates determination of exact location of medical hospital where the one or more medical procedures are taking place. The one or more image capturing units and the one or more audio capturing units are affixed at multiple places to capture the one or more medical procedures efficiently from multiple angles. In an embodiment of the present disclosure, the one or more image capturing units may be one or more video capturing units. For example, the one or more audio capturing units include single or multi-channel distributed microphone on a headset. In an exemplary embodiment of the present disclosure, the one or more image capturing units include one or more infrared video cameras, RGB video camera, time-of-flight camera, one or more Three-Dimensional (3D) sensor for 3D data acquisition or any combination thereof. In an embodiment of the present disclosure, high-resolution stream is stored in a local storage and transmitted to a server in background for storage in a remote database. Also, video stream associated with the one or more videos, or the one or more images are visualized live (in real-time) wirelessly on a monitor or mobile platform by down sampling appropriately via a video BW limiter. In an embodiment of the present disclosure, the one or more videos are processed for low latency, such that important information may be extracted from the processed one or more videos. Further, a plurality of video frames are extracted from each of the one or more videos.

At step 404, one or more medical parameters associated with the one or more medical procedures are extracted from the plurality of video frames, the one or more images or a combination thereof by using a document management-based AI model. In an embodiment of the present disclosure, the document management-based AI model may be a machine learning model. In an exemplary embodiment of the present disclosure, the one or more medical parameters include a set of hand gesture movements of the one or more users, one or more operating tools used by the one or more users and number of the one or more users. In an embodiment of the present disclosure, hand gesture movement details with corresponding associated voice inputs are stored and extracted.

At step 406, one or more vital readings of the patient during the one or more medical procedures are detected by using one or more sensors. In an exemplary embodiment of the present disclosure, the one or more vital readings include blood pressure, pulse, temperature, respiration rate and the like. For example, the one or more sensors include pressure sensor, force sensor, airflow sensor, pulse oximetry, temperature sensor and the like.

At step 408, one or more labels are generated for each of the plurality of video frames, the one or more images, the one or more voice inputs, the extracted one or more medical parameters, the geolocation of one or more users and the detected one or more vital readings based on predefined label information and predefined registration information by using the document management-based AI model. In an embodiment of the present disclosure, the predefined registration information is entered by the one or more users at time of registering the patient and the one or more health professionals before the one or more medical procedures. In an exemplary embodiment of the present disclosure, the predefined registration information include patient name, patient address, patient medical history data, medical procedure details, medical professional details and the like. For example, the medical procedure details include name of the medical procedure, number of medical procedures required to diagnose the patient and the like. For example, the medical professional details include name, designation, type of the one or more health professionals and the like. In an exemplary embodiment of the present disclosure, the type of the one or more health professionals include doctor, nursing staff and the like. In generating the one or more labels for each of the plurality of video frames, the one or more images, the one or more voice inputs, the extracted one or more medical parameters, the geolocation of one or more users and the detected one or more vital readings based on predefined label information and predefined registration information by using the document management-based AI model, the method 400 includes converting the one or more voice inputs into one or more text outputs by using the document management-based AI model. Further, the method 400 includes identifying a set of relevant keywords from the one or more text outputs based on the predefined registration information and the predefined label information by using the document management-based AI model. The method 400 includes identifying speaker of each of the one or more voice inputs based on predefined voice information by using the document management-based AI model. In an embodiment of the present disclosure, the predefined voice information include waveform, behavior characteristics associated with the one or more users and the like. Furthermore, the method 400 includes generating the one or more labels corresponding to the one or more voice inputs based on the identified set of relevant keywords, the identified speaker, the predefined registration information and the predefined label information by using the document management-based AI model. In an embodiment of the present disclosure, the plurality of video frames, the one or more images, the one or more voice inputs and the one or more medical parameters are labelled as points of interest by a strategic two dimensional (2D) labelled marker. This labelling is required to extract the interested point of view at a future date. Further, such data augmentation enables a user to minimize a large video recording to a relatively smaller video file.

At step 410, the generated one or more labels are annotated in the one or more videos and the one or more images by correlating the plurality of video frames, the one or more images, the one or more voice inputs, the extracted one or more medical parameters, the geolocation of one or more users and the detected one or more vital readings based on their corresponding one or more timestamps by using the document management-based AI model. In annotating the generated one or more labels in the one or more videos and the one or more images by correlating the plurality of video frames, the one or more images, the one or more voice inputs, the extracted one or more medical parameters, the geolocation of one or more users and the detected one or more vital readings based on their corresponding one or more timestamps by using the document management-based AI model, the method 400 includes correlating the one or more voice inputs, the extracted one or more medical parameters, the geolocation of one or more users and the detected one or more vital readings with the plurality of video frames and the one or more images based on the one or more timestamps by using the document management-based AI model. Further, the method 400 includes annotating the generated one or more labels in the one or more videos and the one or more images upon performing correlation.

At step 412, a request is received from the one or more users to retrieve one or more specific frames from the annotated one or more videos, a set of specific images from the annotated one or more images or a combination thereof. In an embodiment of the present disclosure, the received request includes one or more keywords corresponding to the plurality of video frames, the one or more images, the one or more voice inputs, the extracted one or more medical parameters, the predefined registration information, the geolocation of one or more users, the detected one or more vital readings or any combination thereof. In an embodiment of the present disclosure, the one or more keywords refer to words describing specific context. For example, when the one or more users require to retrieve a portion of a video in which heart rate of the patient is 110, the one or more users may use “heart rate above 110” as the one or more keywords.

In an embodiment of the present disclosure, the one or more keywords are associated with the one or more labels generated for the one or more voice inputs. For example, when the one or more users require to retrieve a portion of a video in which a doctor said perform the incision, the one or more users may use “perform the incision” as the one or more keywords.

At step 414, the one or more specific frames from the annotated one or more videos, the set of specific images from the annotated one or more images or a combination thereof are retrieved based on the generated one or more labels and the one or more keywords by using the document management-based AI model. In retrieving the one or more specific frames from the annotated one or more videos, the set of specific images from the annotated one or more images or a combination thereof based on the generated one or more labels and the one or more keywords by using the document management-based AI model, the method 400 includes comparing the one or more keywords with the generated one or more labels in the annotated one or more videos, the annotated one or more images or a combination thereof by using the document management-based AI model. Further, the method 400 includes retrieving the one or more specific frames from the annotated one or more videos, the set of specific images from the annotated one or more images or a combination thereof based on result of comparison. The method 400 includes retrieving a set of voice inputs, a set of medical parameters, the geolocation of one or more users and a set of one or more vital readings corresponding to the retrieved one or more specific frames, the retrieved set of specific images or a combination thereof. In an embodiment of the present disclosure, the retrieved one or more specific frames, the retrieved set of specific images or a combination thereof, the retrieved set of voice inputs, the retrieved set of medical parameters, the retrieved geolocation of one or more users and the retrieved set of one or more vital readings are outputted on user interface screen of the one or more electronic devices 108 associated with the one or more users via one or more output formats. For example, the one or more output formats include audio, image, text, video and the like.

In an embodiment of the present disclosure, the method 400 includes extracting and storing the one or more voice inputs and associated recorded video reference in the storage unit 206. The one or more voice inputs may be associated with one or more operating doctors. In an embodiment of the present disclosure, the method 400 includes searching for a video frame or image in order to provide an image or a video query. The method 400 includes performing down-samples data and segmentation on the one or more images or the plurality of video frames to detect point of interest in the one or more images or the plurality of video frames.

At step 416, the retrieved the one or more specific frames, the set of specific images or a combination thereof on user interface screen of one or more electronic devices 108 associated with the one or more users. In an exemplary embodiment of the present disclosure, the one or more electronic devices 108 may include a laptop computer, desktop computer, tablet computer, smartphone, wearable device, smart watch, a digital camera and the like. In an embodiment of the present disclosure, outputting the one or more specific frames, the set of specific images or a combination thereof on user interface screen of the one or more electronic devices 108 enables easy detection without need for going through whole captured video.

In an embodiment of the present disclosure, before using the document management-based AI model for performing multiple operations, such as label generation, annotation and the like, the document management-based AI model is required to be trained. Further, the document management-based AI model is pretrained on a dataset of such operating procedure videos with commentary metadata and mixture of supervised and unsupervised video data training. In an embodiment of the present disclosure, tools are annotated in training video and hand pose detections are refined using existing models. Thus, this step drastically improves search speeds through huge database of videos.

Further, the method 400 includes determining one or more analytics parameters associated with the one or more medical procedures by analyzing the medical data using the document management-based AI model. In an embodiment of the present disclosure, the one or more analytics parameters include time consumed in each stage of the one or more medical procedures, response of the patient to certain operating events and the like. In an embodiment of the present disclosure, time consumed in each stage of the one or more medical procedures is associated with the one or more vital readings of the patient to ascertain patient response to certain events. For example, response of the patient to certain operation event may be fluctuation in heart rate, sudden decrease in SpO2 level and the like upon performing incision. Further, the method 400 includes outputting the determined one or more analytics parameters on user interface screen of the one or more electronic devices 108 associated with the one or more users. In an embodiment of the present disclosure, the one or more users may also retrieve one or more portions of the one or more videos, the one or more images or a combination thereof corresponding to the determined one or more analytics parameters by using specific keywords related to the determined one or more analytics parameters. In an embodiment of the present disclosure, analytics associated to doctor's commentary, recorded visuals and patient vitals are also presented in real time.

The AI-based method 400 may be implemented in any suitable hardware, software, firmware, or combination thereof.

Thus, various embodiments of the present AI-based computing system 104 provide a solution to document medical procedures. The AI-based computing system 104 extracts the sequence of interest from a long medical procedure based on defined context or stage in the procedure quickly. In an embodiment of the present disclosure, the AI-based computing system 104 not only help in quick evaluation but also in training new medical practitioners. The AI-based computing system 104 helps to derive insights from a procedure via analytics as the association between clinician's commentary, recorded visuals and patient vitals are disjoint. Further, the AI-based computing system 104 further enables deriving insights from multiple procedures via analytics as well identified synchronization points between various events in the recorded sessions do not exist. The AI-based computing system 104 system provides ability to train trainees with the documented footage in a digital or virtual setting. Doctor may get a multi perspective video stream or video in 3D for augmented reality and virtual reality if multiple cameras are present. By model fitting the patient with a 3D model, a complete immersive 3D experience is possible. The present system may be employed with any procedure with hierarchical subprocesses, such as pilot training, medical, dental, training, court cases and the like.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at least one processor or central processing unit (CPU). The CPUs are interconnected via system bus 208 to various devices such as a random-access memory (RAM), read-only memory (ROM), and an input/output (I/O) adapter. The I/O adapter can connect to peripheral devices, such as disk units and tape drives, or other program storage devices that are readable by the system. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

We claim:

1. An Artificial Intelligence (AI) based computing system for documenting medical procedures, the AI-based computing system comprising:
one or more hardware processors; and
a memory coupled to the one or more hardware processors, wherein the memory comprises a plurality of modules in the form of programmable instructions executable by the one or more hardware processors, and wherein the plurality of modules comprises:
a medical data receiver module configured to receive medical data associated with one or more medical procedures from one or more data capturing units, wherein the medical data comprises at least one of: one or more videos, one or more images of one or more medical procedures, one or more voice inputs, and geolocation of one or more users, and wherein a plurality of video frames are extracted from each of the one or more videos by using a frame extraction technique;
a medical parameter extraction module configured to extract one or more medical parameters associated with the one or more medical procedures from at least one of: the plurality of video frames and the one or more images by using a document management-based AI model;
a vital detection module configured to detect one or more vital readings of a patient during the one or more medical procedures by using one or more sensors, wherein the one or more vital readings comprises at least one of: blood pressure, pulse, temperature, and respiration rate;
a label generation module configured to generate one or more labels for each of: the plurality of video frames, the one or more images, the one or more voice inputs, the extracted one or more medical parameters, the geolocation of one or more users, and the detected one or more vital readings based on predefined label information and predefined registration information by using the document management-based AI model;
an annotation module configured to annotate the generated one or more labels in the one or more videos and the one or more images by correlating the plurality of video frames, the one or more images, the one or more voice inputs, the extracted one or more medical parameters, the geolocation of one or more users, and the detected one or more vital readings based on their corresponding one or more timestamps by using the document management-based AI model;
a request receiver module configured to receive a request from the one or more users to retrieve at least one of: one or more specific frames from the annotated one or more videos and a set of specific images from the annotated one or more images, wherein the received request comprises: one or more keywords corresponding to at least one of: the plurality of video frames, the one or more images, the one or more voice inputs, the extracted one or more medical parameters, the predefined registration information, the geolocation of one or more users, and the detected one or more vital readings;
a data retriever module configured to retrieve at least one of: the one or more specific frames from the annotated one or more videos and the set of specific images from the annotated one or more images based on the generated one or more labels and the one or more keywords by using the document management-based AI model; and
a data output module configured to output the retrieved at least one of: the one or more specific frames and the set of specific images on a user interface screen of one or more electronic devices associated with the one or more users.

2. The AI-based computing system of claim 1, wherein the one or more data capturing units comprises: one or more image capturing units, one or more audio capturing units, and one or more Global Positioning System (GPS) units, and wherein the one or more audio capturing units comprise one of: single and a multi-channel distributed microphone on a headset.

3. The AI-based computing system of claim 2, wherein the one or more image capturing units comprises at least one of: one or more infrared video cameras, a Red Green Blue (RGB) video camera, a time-of-flight camera, and one or more Three-Dimensional (3D) sensors for 3D data acquisition.

4. The AI-based computing system of claim 1, wherein in generating the one or more labels for each of: the plurality of video frames, the one or more images, the one or more voice inputs, the extracted one or more medical parameters, the geolocation of one or more users, and the detected one or more vital readings based on the predefined label information, and the predefined registration information by using the document management-based AI model, the label generation module is configured to:
convert the one or more voice inputs into one or more text outputs by using the document management-based AI model;
identify a set of relevant keywords from the one or more text outputs based on the predefined registration information and the predefined label information by using the document management-based AI model;
identify a speaker of each of the one or more voice inputs based on predefined voice information by using the document management-based AI model; and
generate the one or more labels corresponding to the one or more voice inputs based on the identified set of relevant keywords, the identified speaker, the predefined registration information, and the predefined label information by using the document management-based AI model.

5. The AI-based computing system of claim 1, wherein in annotating the generated one or more labels in the one or more videos and the one or more images by correlating the plurality of video frames, the one or more images, the one or more voice inputs, the extracted one or more medical parameters, the geolocation of one or more users and the detected one or more vital readings based on their corresponding one or more timestamps by using the document management-based AI model, the annotation module is configured to:
correlate the one or more voice inputs, the extracted one or more medical parameters, the geolocation of one or more users and the detected one or more vital readings with the plurality of video frames and the one or more images based on the one or more timestamps by using the document management-based AI model;
annotate the generated one or more labels in the one or more videos and the one or more images upon performing correlation.

6. The AI-based computing system of claim 1, wherein the one or more users are one or more health professionals performing the one or more medical procedures.

7. The AI-based computing system of claim 1, wherein the one or more medical parameters comprises: a set of hand gesture movements of the one or more users, one or more operating tools used by the one or more users, and number of the one or more users.

8. The AI-based computing system of claim 1, wherein the predefined registration information comprises: patient name, patient address, patient medical history data, medical procedure details, and medical professional details.

9. The AI-based computing system of claim 1, wherein in retrieving at least one of: the one or more specific frames from the annotated one or more videos and the set of specific images from the annotated one or more images based on the generated one or more labels and the one or more keywords by using the document management-based AI model, the data retriever module:
compares the one or more keywords with the generated one or more labels in at least one of: the annotated one or more videos and the annotated one or more images by using the document management-based AI model;
retrieves at least one of: the one or more specific frames from the annotated one or more videos and the set of specific images from the annotated one or more images based on result of comparison; and
retrieves a set of voice inputs, a set of medical parameters, the geolocation of one or more users, and a set of one or more vital readings corresponding to retrieved at least one of: the one or more specific frames and the set of specific images, wherein the retrieved at least one of: the one or more specific frames and the set of specific images, the retrieved set of voice inputs, the retrieved set of medical parameters, the retrieved geolocation of one or more users, and the retrieved set of one or more vital readings are outputted on user interface screen of the one or more electronic devices associated with the one or more users via one or more output formats and wherein the one or more output formats comprises: audio, image, text, and video.

10. The AI-based computing system of in claim 1, further comprises an analytics determination module configured to:
determine one or more analytics parameters associated with the one or more medical procedures by analyzing the medical data using the document management-based AI model, wherein the one or more analytics parameters comprises: time consumed in each stage of the one or more medical procedures and response of patient to certain operating events; and
output the determined one or more analytics parameters on user interface screen of the one or more electronic devices associated with the one or more users.

11. An Artificial Intelligence (AI) based method for documenting medical procedures, the AI-based method comprising:
receiving, by one or more hardware processors, medical data associated with one or more medical procedures from one or more data capturing units, wherein the medical data comprises at least one of: one or more videos, one or more images of one or more medical procedures, one or more voice inputs and geolocation of one or more users, and wherein a plurality of video frames are extracted from each of the one or more videos by using a frame extraction technique;
extracting, by the one or more hardware processors, one or more medical parameters associated with the one or more medical procedures from at least one of: the plurality of video frames and the one or more images by using a document management-based AI model;
detecting, by the one or more hardware processors, one or more vital readings of a patient during the one or more medical procedures by using one or more sensors, wherein the one or more vital readings comprises: blood pressure, pulse, temperature, and respiration rate;
generating, by the one or more hardware processors, one or more labels for each of: the plurality of video frames, the one or more images, the one or more voice inputs, the extracted one or more medical parameters, the geolocation of one or more users and the detected one or more vital readings based on predefined label information and predefined registration information by using the document management-based AI model;
annotating, by the one or more hardware processors, the generated one or more labels in the one or more videos and the one or more images by correlating the plurality of video frames, the one or more images, the one or more voice inputs, the extracted one or more medical parameters, the geolocation of one or more users and the detected one or more vital readings based on their corresponding one or more timestamps by using the document management-based AI model;
receiving, by the one or more hardware processors, a request from the one or more users to retrieve at least one of: one or more specific frames from the annotated one or more videos and a set of specific images from the annotated one or more images, wherein the received request comprises: one or more keywords corresponding to at least one of: the plurality of video frames, the one or more images, the one or more voice inputs, the extracted one or more medical parameters, the predefined registration information, the geolocation of one or more users and the detected one or more vital readings;
retrieving, by the one or more hardware processors, at least one of: the one or more specific frames from the annotated one or more videos and the set of specific images from the annotated one or more images based on the generated one or more labels and the one or more keywords by using the document management-based AI model; and
outputting, by the one or more hardware processors, the retrieved at least one of: the one or more specific frames and the set of specific images on user interface screen of one or more electronic devices associated with the one or more users.

12. The AI-based method of claim 11, wherein the one or more data capturing units comprises: one or more image capturing units, one or more audio capturing units, and one or more Global Positioning System (GPS) units, wherein the one or more audio capturing units comprises one of a single and multi-channel distributed microphone on a headset.

13. The AI-based method of claim 12, wherein the one or more image capturing units comprises at least one of: one or more infrared video cameras, a Red Green Blue (RGB) video camera, a time-of-flight camera, and one or more Three-Dimensional (3D) sensors for 3D data acquisition.

14. The AI-based method of claim 11, wherein generating the one or more labels for each of: the plurality of video frames, the one or more images, the one or more voice inputs, the extracted one or more medical parameters, the geolocation of one or more users and the detected one or more vital readings based on the predefined label information and the predefined registration information by using the document management-based AI model comprises:
converting the one or more voice inputs into one or more text outputs by using the document management-based AI model;
identifying a set of relevant keywords from the one or more text outputs based on the predefined registration information and the predefined label information by using the document management-based AI model;
identifying speaker of each of the one or more voice inputs based on predefined voice information by using the document management-based AI model; and
generating the one or more labels corresponding to the one or more voice inputs based on the identified set of relevant keywords, the identified speaker, the predefined registration information and the predefined label information by using the document management-based AI model.

15. The AI-based method of claim 11, wherein annotating the generated one or more labels in the one or more videos and the one or more images by correlating the plurality of video frames, the one or more images, the one or more voice inputs, the extracted one or more medical parameters, the geolocation of one or more users, and the detected one or more vital readings based on their corresponding one or more timestamps by using the document management-based AI model comprises:
correlating the one or more voice inputs, the extracted one or more medical parameters, the geolocation of one or more users, and the detected one or more vital readings with the plurality of video frames and the one or more images based on the one or more timestamps by using the document management-based AI model; and
annotating the generated one or more labels in the one or more videos and the one or more images upon performing the correlation.

16. The AI-based method of claim 11, wherein the one or more users are one or more health professionals performing the one or more medical procedures.

17. The AI-based method of claim 11, wherein the one or more medical parameters comprises: a set of hand gesture movements of the one or more users, one or more operating tools used by the one or more users, and number of the one or more users.

18. The AI-based method of claim 11, wherein the predefined registration information comprises: patient name, patient address, patient medical history data, medical procedure details, and medical professional details.

19. The AI-based method of claim 11, wherein retrieving at least one of: the one or more specific frames from the annotated one or more videos and the set of specific images from the annotated one or more images based on the generated one or more labels and the one or more keywords by using the document management-based AI model:
comparing the one or more keywords with the generated one or more labels in at least one of: the annotated one or more videos and the annotated one or more images by using the document management-based AI model;
retrieving at least one of: the one or more specific frames from the annotated one or more videos and the set of specific images from the annotated one or more images based on result of comparison; and
retrieving a set of voice inputs, a set of medical parameters, the geolocation of one or more users and a set of one or more vital readings corresponding to retrieved at least one of: the one or more specific frames and the set of specific images, wherein the retrieved at least one of: the one or more specific frames and the set of specific images, the retrieved set of voice inputs, the retrieved set of medical parameters, the retrieved geolocation of one or more users and the retrieved set of one or more vital readings are outputted on user interface screen of the one or more electronic devices associated with the one or more users via one or more output formats and wherein the one or more output formats comprises: audio, image, text and video.

20. The AI-based method of claim 11, further comprises:
determining one or more analytics parameters associated with the one or more medical procedures by analyzing the medical data using the document management-based AI model, wherein the one or more analytics parameters comprises: time consumed in each stage of the one or more medical procedures and response of patient to certain operating events; and
outputting the determined one or more analytics parameters on user interface screen of the one or more electronic devices associated with the one or more users.
